(19) United States
(12) Patent Application Publication
(10) Pub. No.: US 2007/0021645 A1
(43) Pub. Date: Jan. 25, 2007

(54) Medical device

(76) Inventor: Joachim Zimmerman, Wasserburg (DE)

Correspondence Address: LAMBERT & ASSOCIATES, P.L.L.C., 92 STATE STREET, BOSTON, MA, 02109-2004, US

(21) Appl. No.: 10/564,774

(22) PCT Filed: Jul. 13, 2004
(86) PCT No.: PCT/EP04/07723

§ 371 Date: Jun. 19, 2006

(30) Foreign Application Priority Data
Jul. 16, 2003 (DE) 103 32 475.5

(51) Int. Cl. A61N 2/04

(52) U.S. Cl. 600/14

(57) ABSTRACT

In the case of a medical apparatus, for the treatment of living beings, having a magnetic field, more especially having a pulsating magnetic field, an element which radiates a magnetic field is to be inserted into a bodily orifice of the living being.

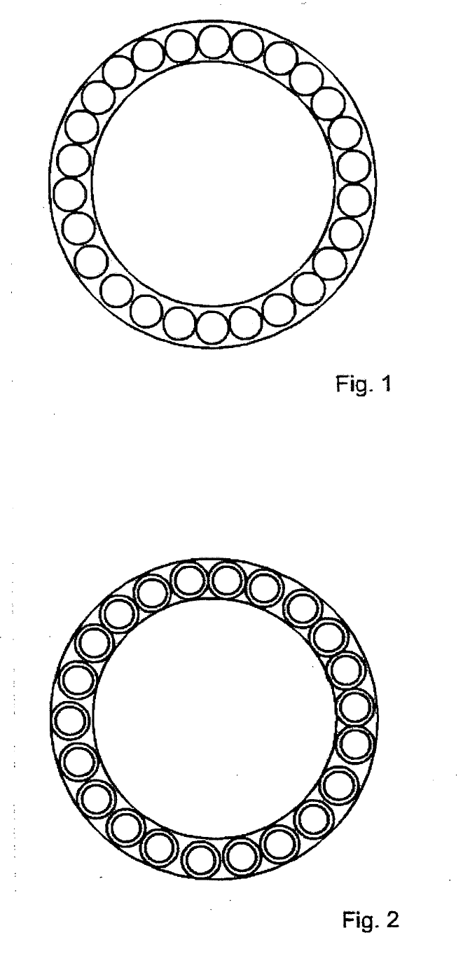

The invention relates to a medical apparatus, for the treatment of living beings, having a magnetic field, more especially having a pulsating magnetic field.

### PRIOR ART

In recent years, therapy systems having magnetic fields, more especially having pulsating magnetic fields (PEMF), have found scientific recognition and very great approval by the patients. Appropriate devices and apparatuses are used, above all, in the therapy of painful conditions which are caused by excessive degeneration (=wear), for example joint pains. Furthermore, they are used in the case of abnormal blood circulation, for example in the case of Morbus Sudeck, Raynaud's disease and abnormal blood circulation caused by diabetes.

Appropriate devices are generally offered with a large-sized field coil, in which a patient is placed with his whole body. Furthermore, there are also pulse generators with flat applicators, which generate a magnetic stray field which fully penetrates the body. It is identical, in respect of frequency and intensities, to the pulsating magnetic field of the large-sized field coil. However, the concentric magnetic field of the large-sized field coil is more comprehensive.

### OBJECT

The basic object of the present invention is to broaden the use of magnetic field therapy.

### ACHIEVEMENT OF THE OBJECT

This object is achieved when an element, which radiates a magnetic field, is inserted into a vagina of a woman or into an anal region of a living being, the element being flexible and comprising an inert tube, in which magnetised or magnetisable balls are introduced in an attractive alignment, or magnetised or magnetisable rings are arranged in adjacent rows in opposite alignment, and which tube is shaped to form a ring.

This means that the magnetic field therapy not only exerts an influence upon the body of the living being from externally, but radiates directly into the interior, without having to traverse the outer skin. In practice, it has become evident that a substantially more direct influence upon the internal organs can be achieved hereby.

Unclear hypogastrium complaints, period pains and premenstrual syndromes have been able to be relieved with the element which radiates the magnetic field. Furthermore, the loss of libido was able to be counteracted.

It is to be assumed therefrom that a similarly positive effect will also be observed with the insertion of the element into the anal region. Here, in the case of men, it could be used more especially for the relief of prostate complaints and for a positive influence on erection malfunctions.

It is advisable to give the element an annular configuration, since the element, radiating a magnetic field, is inserted into the vagina. For hygienic reasons, the element is provided with an inert coating.

Particularly positive observations were made with flexibly configured elements. In such case, it has been proposed, for the sake of simplicity, to fill the inert tube with the magnetised or magnetisable balls and to shape it to form a ring, which is then inserted into the vagina, for example. Many possibilities are conceivable here and are to be covered by the present invention.

The annular configuration of the radiating element has the particular advantage that the radiated magnetic field is very harmonic. The arrangement of the small magnetic rings in adjacent rows in opposite alignment produces an intense field of energy and a vitalisation of the body. If, however, the small magnetic balls in an attractive alignment are shaped to form a ring, this produces a calming, harmonising effect.

1. Medical apparatus, for the treatment of living beings, having a magnetic field, more especially having a pulsating magnetic field, characterised in that an element, which radiates a magnetic field, is inserted into a vagina of a woman or into an anal region of a living being, the element being flexible and comprising an inert tube, into which magnetised or magnetisable balls are introduced in an attractive alignment, or magnetised or magnetisable rings are arranged in adjacent rows in opposite alignment, and which tube is shaped to form a ring.
